(19) United States
(12) Patent Application Publication
(10) Pub. No.: US 2024/0226301 A9
(43) Pub. Date: Jul. 11, 2024

(54) ANHYDROUS ORAL PHARMACEUTICAL SUSPENSIONS

(71) Applicant: Professional Compounding Centers of America, Ltd., Houston, TX (US)

(72) Inventors: Daniel BANOV, Sugarland, TX (US); Connie HWEE, Sugarland, TX (US); Yi LIU, Cypress, TX (US)

(21) Appl. No.: 18/485,090

(22) Filed: Oct. 11, 2023

Related U.S. Application Data
(60) Provisional application No. 63/417,528, filed on Oct. 19, 2022.

(51) Int. Cl. A61K 47/14; A61K 45/06; A61K 47/44; A61K 47/46

(52) U.S. Cl. CPC A61K 47/14; A61K 45/06; A61K 47/44; A61K 47/46

(57) ABSTRACT

Compositions and methods of using and manufacturing an oral pharmaceutical anhydrous suspension are provided. The anhydrous suspension is suitable to suspend active pharmaceutical ingredients (APIs), improve the stability of oral pharmaceutical suspensions, and help in the formation of a thermo-reversible gel and shear thinning to keep the APIs suspended. The anhydrous suspension disclosed herein may include glyceryl distearate NF (e.g., in an amount of from about 0.1 wt. % to about 10 wt. %) and stearoyl polyoxyl-32 glycerides NF (e.g., in an amount of from about 0.1 wt. % to about 10 wt. %). In embodiments, the anhydrous suspension also includes glycerin (e.g., in an amount of about 5 wt. %). The anhydrous suspension may also include one or more of medium chain triglycerides NF, vitamin E acetate (DL) USP liquid (1 IU/mg), flavoring agent (e.g., a sweeter or other flavoring agent), ascorbyl palmitate NF, and other components.

### CROSS REFERENCE TO RELATED APPLICATIONS

This application claims priority to U.S. Provisional Patent Application No. 63/417,528, filed on Oct. 19, 2022, titled “ANHYDROUS ORAL PHARMACEUTICAL SUSPENSIONS”; which is herein incorporated by reference.

### FIELD OF THE INVENTION

The present disclosure relates to pharmaceutical suspending agents that improve stability in oral anhydrous pharmaceutical suspensions. Specifically, the anhydrous pharmaceutical suspending agent discussed herein includes glyceryl distearate NF and stearoyl polyoxyl-32 glycerides NF.

### BACKGROUND

Some active ingredients are insoluble in all acceptable media and, therefore, may need to be administered as a tablet, capsule, or as a suspension. In some situations, suspensions possess certain advantages over other dosage forms. Because of their liquid character, suspensions represent an ideal dosage form for patients who have difficulty swallowing tablets or capsules. This factor is of particular importance in the administration of drugs to children and elderly persons. Additionally, suspensions of insoluble drugs can also be used externally, often as protective agents. Even more, anhydrous suspensions allow for compounding of hydrophilic and lipophilic ingredients, water-unstable active pharmaceutical ingredients (APIs), or where aqueous stability of a drug in suspension is unknown.

One challenge of anhydrous pharmaceutical suspensions is the thixotropy effect. All suspensions have thixotropic property. Thixotropy is the property of certain fluids and gels of becoming thinner when a constant force is applied. For example, thixotropy is a property of anhydrous pharmaceutical suspensions changing viscosity and become more fluid when subject to a shear force, such as mixing. The longer the fluid is under shear stress, the less its viscosity. After reduction of the force, the viscosity recovers fully to the initial state in an appropriate period of time. The ingredients used to increase viscosity of oils for oral use generally do not have the thixotropy property required for oral suspensions.

Another challenge of anhydrous pharmaceutical suspensions includes a common problem for all types of suspensions—sedimentation, caking, distribution, and re-suspension of the solid particles. A suspension should not settle rapidly and it should be sufficiently fluid to flow easily under the conditions of administration. Because suspensions are energetically unstable, the particles that have settled tend to interact to form a cake or hard crystalline network. To prevent this, it is beneficial to formulate suspensions such that caking is minimized so particles that have settled can be readily re-dispersed upon shaking.

### SUMMARY

Compositions and methods are provided for an oral pharmaceutical anhydrous suspension that is suitable to suspend active pharmaceutical ingredients (APIs). The disclosed compositions and methods improve the stability of oral pharmaceutical suspensions, such as, for example, by improving the formation of a thermo-reversible gel and shear thinning that keeps APIs suspended within oral pharmaceutical suspensions. For example, as discussed in more detail herein, the disclosed compositions and methods provide for improvements to the uniformity of the pharmaceutical anhydrous suspension as well as making the suspension more suitable for patient intake.

In embodiments, this particular suspension may include glyceryl distearate NF, stearoyl polyoxyl-32 glycerides NF, medium chain triglycerides NF, vitamin E acetate (DL) USP liquid (1 IU/mg), PUREFRUIT™ select, ascorbyl palmitate NF, glycerin USP (natural), another component, or one or more combinations thereof. Additionally, this suspension can enable rapid dispersion of APIs (e.g., when treating a human patient) and can reduce sedimentation of anhydrous pharmaceutical suspensions. This suspension can also improve chemical, physical, and microbiologic stability of the anhydrous pharmaceutical without affecting the therapeutically effective API. Further, in some embodiments, the synergistic blend of stearoyl polyoxyl-32 glycerides and glycerin distearate can be used to suspend suitable APIs for improvement of the stability of an anhydrous pharmaceutical suspension, improvement to anti-flocculation properties assisting with the homogeneity of the anhydrous pharmaceutical suspension, and improvement to texture and/or taste of oral anhydrous pharmaceutical suspensions.

In some embodiments, the anhydrous suspension may comprise glyceryl distearate in an amount of from about 0.1 wt. % to about 10 wt. %, stearoyl polyoxyl-32 glycerides in an amount of from about 0.1 wt. % to about 10 wt. %, and one or more active pharmaceutical ingredients (APIs). In one example aspect of this embodiment, the anhydrous suspension can include stearoyl polyoxyl-32 glycerides in an amount of about 3 wt. % and glyceryl distearate in an amount of about 3 wt. %. In some embodiments, the anhydrous suspension also includes glycerin (e.g., in an amount of about 5 wt. %). In some embodiments, the anhydrous suspension also includes medium chain triglycerides (MCTs) (e.g., in an amount of from about 40 wt. % to about 99.5 wt. %). In some embodiments, the anhydrous suspension also includes flavoring agents.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations of the present disclosure are described in detail below with reference to the attached drawing figures, wherein:

FIG. 1 illustrates particular physical characteristics associated with particle suspension of the oral pharmaceutical anhydrous suspension disclosed herein, in accordance with embodiments herein;

FIG. 2 depicts an example graph illustrating additional particular physical characteristics of the oral pharmaceutical anhydrous suspension associated with thixotropy, uniform emulsion, and homogeneity of the suspension, in accordance with embodiments herein;

FIGS. 3-5 depict another test that helps illustrate other particular physical characteristics of the oral pharmaceutical anhydrous suspension related to self-emulsifying properties of the suspension, in accordance with embodiments herein;

FIGS. 6-7 depict other particular physical characteristics of the oral pharmaceutical anhydrous suspension related to drug residue reduction throughout nasogastric tubes, in accordance with embodiments herein; and

FIG. 8 depicts an example flowchart for a method of manufacturing and using an anhydrous suspension, in accordance with embodiments herein.

### DETAILED DESCRIPTION

Aspects provided herein are directed to a novel combination of components to form an oral pharmaceutical anhydrous suspension suitable to suspend active pharmaceutical ingredients (APIs), improve the stability of oral pharmaceutical suspensions, and help in the formation of a thermo-reversible gel and shear thinning necessary to keep APIs suspended within oral pharmaceutical suspensions. The synergistic blend of stearoyl polyoxyl-32 glycerides and glyceryl distearate has unique anti-flocculation and thixotropic properties, which improve the homogeneity of the oral pharmaceutical anhydrous suspensions. Oral anhydrous pharmaceutical suspensions, comprising the synergistic blend of stearoyl polyoxyl-32 glycerides and glyceryl distearate, may also comprise an oil, such as medium chain triglycerides (MCT) oil. Additionally or alternatively, this synergistic blend may also comprise one or more additional components, such as, for example APIs, sweeteners (e.g., PUREFRUIT™ select), other flavoring agents, medium chain triglycerides (MCTs), vitamin E acetate, ascorbyl palmitate NF, glycerin USP (natural), among other components.

This synergistic blend can improve or enhance the intake of a pre-determined amount of API when this pharmaceutical suspension is administered to patients (e.g., based at least in part on the viscosity of this oral pharmaceutical anhydrous suspension). Further, the technology disclosed can provide for improved physical characteristics of oral pharmaceutical anhydrous suspensions that are suitable for suspending APIs, broader applications for the administration of APIs, and a longer default beyond use dates (BUDs). The technology disclosed herein can also be suitable for APIs that are either unstable in water or have incompatibilities with existing aqueous vehicles. Further, this disclosure provides for a self-emulsifying drug delivery system and compatibility of an oral pharmaceutical anhydrous suspension with nasogastric feeling tubes, which are described in more detail herein.

In embodiments, the anhydrous suspension, self-emulsifying drug delivery system, and methods described herein provide for increased drug solubility, increased dispersibility, increased absorption, and increased bioavailability. As an example, the method for the anhydrous suspension includes combining surfactants within an anhydrous oral vehicle that is easily mixable with water or juice, for example, without the need of additional surfactants. The self-emulsifying properties of the anhydrous suspension can provide for improved dispersibility of water-soluble drugs and the release of lipophilic drugs, for example. By way of illustration, this oral pharmaceutical anhydrous suspension can improve or enhance the release of the one or more APIs such that substantially all of the API (e.g., about ninety percent or greater) is released into a gastrointestinal tract of a human patient (e.g., within less than one hour following oral administration) for absorption into the systemic circulation of the gastrointestinal tract. As another example, this oral pharmaceutical anhydrous suspension can improve the release of the one or more APIs such that a desired rate of the release of the APIs following oral administration at a certain location of the gastrointestinal tract is achieved.

As used herein, “wt. %” means the percent concentration of the component in the formulation measured on a weight-to-weight basis. For example, 1 wt. % of component A=[(mass of component A)/(total mass of the anhydrous suspension including the mass of component A)]×100.

As used herein, the term “component” can mean one substance or a mixture of substances. As will be appreciated, some components of the anhydrous suspension described herein can possess multiple functions. For example, a given component may act as both an increasing solubility and an increasing dispersibility agent. In some such instances, the function of a given component can be considered singular, even though its properties may allow multiple functionalities. In some embodiments, each component of the anhydrous suspension can comprise a different component or mixture of components.

Reference to “about” a value or parameter herein refers to the usual error range for the respective value readily known to the skilled person in this technical field.

“Stearoyl polyoxyl-32 glycerides” (e.g., having the chemical formula C₁₈H₃₆O₂.x(C₂H₄O)ₙ(C₂H₄O)ₙ(C₂H₄O)ₙC₃H₈O₃) have the ability to self-emulsify on contact with an aqueous media to form a fine dispersion (i.e., microemulsion (SMEDDS)).

“Glyceryl distearate” (e.g., having the chemical formula C₃₉H₇₆O₅) is a white or off-white waxy mass or powder. Glyceryl distearate is a diester of glycerin and stearic acid and results in a mixture of mono-, di-, and triglyceride substances. Glyceryl distearate is often used in cosmetic products as an emollient, thickening agent, and emulsifier.

As used herein, the term “flavoring agent” may be a taste masking agent or another type of pharmaceutically compatible flavoring agent, for example. Stated differently, the term “flavoring agent” may refer to any food-grade material that may be added to or present in an orally consumable product to provide a desired flavor. For example, the flavoring agent may include a natural, artificial, or combination thereof. The flavoring agent may include a synthetic oil, a synthetic flavoring aromatic, another type of oil or aromatic flavoring agent, an oleo resin or extract derived from a plant, leaf, flower, fruit, vegetable, etc. Some examples of a flavoring agent may include, for example, a natural sweetener (e.g., a Monk fruit sweetener, a steviol glycoside), acesulfame potassium, aspartame, sucralose, thaumatin, a raspberry flavoring agent, a lemon flavoring agent, a cherry flavoring agent, a vanilla flavoring agent, another type of flavoring agent, or one or more combinations thereof.

The term “glycerin” may refer to glycerol, propane-1,2,3-triol, or another type of simple polyol compound (e.g., typically derived from plant or animal sources where it occurs in triglycerides or esters of glycerol with long-chain carboxylic acids). For example, glycerin can be used as a solvent, sweetener, preservative, thickening agent, and lubricant, among other things.

The term “active pharmaceutical ingredient” (API) refers to a component or a compound that induces a particular or intended effect in diagnosis, cure, mitigation, disease prevention or treatment, or has an effect in restoring, correcting, or modifying physiological functions of a human, animal, or other living creature.

The term “treatment” or “therapeutic effect” refers to the reduction in severity or frequency of a symptom, or its underlying cause, or the prevention of the occurrence of a symptom or its underlying cause (e.g., including providing protection or support to human or animal tissue over a duration of time).

The term “medium chain triglycerides (MCT)” may be any type of oil, including, for example, MCT oil or vegetable oil.

As used herein, the singular form “a”, “an,” and “the” can include plural references, unless indicated otherwise.

As used herein, the term “or” includes the conjunctive, the disjunctive, and both (a or b thus includes either a or b, as well as a and b).

For purposes of this disclosure, the word “including” or “having” has the same broad meaning as the word “comprising.”

By way of background, pharmaceutical suspensions include solid particles (e.g., of variable sizes) dispersed in a liquid medium, generally an aqueous solution. Typically, pharmaceutical suspensions include a suspending agent that helps the APIs and other components stay suspended in the liquid medium, thereby reducing caking at the bottom of the suspension. These suspending agents are helpful in that they can maintain a consistency of the solid particles throughout the suspending medium with the API as solid particles staying suspended in the continuous phase, thereby allowing consistent withdrawal of uniform doses. One of the properties of a well-formulated suspension, suitable for a therapeutic use when administered to a human patient, is that it should easily be re-suspended by the use of moderate agitation (e.g., shaking). A suitable suspension allows for the withdrawal of uniform and accurate doses during or throughout the period of medication.

Prior anhydrous suspension forms have encountered stability problems associated with maintaining the APIs in suspension. Stability problems include sedimentation, creaming, crystal growth (agglomeration), separation, and difficulty to re-disperse to obtain original suspensions. For example, may prior oral pharmaceutical suspensions enable the APIs to settle out as either a sediment or by creaming to the surface, thereby resulting in variations in the therapeutic concentration of APIs within the oral suspension. As such, this results in under dosing or overdosing of the patient, which may seriously compromise the patient's recovery.

Additionally, oral pharmaceutical suspensions should be readily pourable so that the dose is easy to administer. Oral pharmaceutical suspensions that should be readily pourable effectively places an upper limit on the viscosity of the suspensions. This upper limit viscosity restriction also limits the amount of APIs that the overall composition will suspend. For example, highly viscous suspensions are problematic for subcutaneous administration because viscous solutions do not flow easily from a syringe through a needle. For subcutaneous administration, the limit to the acceptable viscosity of formulations is usually approximately 20-25 millipascal seconds (assuming the use of a typical needle size being thinner than 27 gauge). Anhydrous pharmaceutical suspensions with higher viscosities can cause pain at the site of injection and potentially require injection forces too high for common syringes to withstand. Further, the drug may even not be administrable through the syringe at these higher viscosities.

As such, it is desirable to formulate oral anhydrous pharmaceutical suspensions that reduce sedimentation, creaming, crystal growth, and separation of APIs from the suspension. Further, it is desirable to formulate oral anhydrous pharmaceutical suspensions that reduce the difficulty to re-disperse APIs within the suspension to allow for the withdrawal of uniform and accurate doses during or throughout the period of medication. Furthermore, it is desirable to formulate oral anhydrous pharmaceutical suspensions with a viscosity that is below the upper limit viscosity, such that the oral anhydrous pharmaceutical suspension is readily pourable and easy to administer without affecting the desired therapeutic effect of the API.

Embodiments of the present disclosure can reduce sedimentation, creaming, crystal growth, and separation of APIs from the suspension and reduce the difficulty to re-disperse APIs within the suspension. Further, embodiments of the present disclosure provides oral anhydrous pharmaceutical suspensions that have a viscosity below an upper limit viscosity for administration. Embodiments of the present disclosure may include an anhydrous suspension having glyceryl distearate in an amount of from about 0.1 wt. % to about 10 wt. %, stearoyl polyoxyl-32 glycerides in an amount of from about 0.1 wt. % to about 10 wt. %, and one or more active pharmaceutical ingredients (APIs). In some embodiments, the oral pharmaceutical suspensions described herein may comprise oil (anhydrous) solutions that may include natural sweeteners, such as Monk fruit sweetener, among other suitable ingredients. The synergistic blend of glyceryl distearate and stearoyl polyoxyl-32 glycerides can influence the physical stability of anhydrous suspensions (e.g., rather than the mere physical characteristics of the APIs themselves affecting the overall physical stability of the anhydrous suspension).

Stearoyl macrogol-32 glycerides are nonionic water-dispersible surfactants for lipid-based formulations, and glycerol distearate is a glyceride with an intermediate melting point that can assist with lubrication, can aid in the flow of powder blends (e.g., for capsule filling), and can also act as a coating agent (e.g., for taste masking). The synergistic blend of glyceryl distearate and stearoyl polyoxyl-32 glycerides can increase oral bioavailability of poorly water-soluble APIs. In addition, this synergistic blend can self-emulsify in aqueous media, forming a fine dispersion (e.g., microemulsion). This self-emulsifying drug delivery system provides an effective approach for delivery of poorly water soluble, lipophilic APIs. Surfactive, dispersible, and oily excipients of the synergistic blend can also improve oral bioavailability by a number of mechanisms, including, but not limited to: enabling higher drug loading by increasing the wettability/solubility of API in the formulation; maintaining the API in a solubilized state in gastro-intestinal fluid promoting increased absorption; and facilitating selective absorption of certain APIs via the lymphatic transport system.

In some aspects, the combination of stearoyl polyoxyl-32 glycerides and glyceryl distearate produces a particular synergistic matrix complex that provides a unique thixotropic flow (e.g., it thins as it is shaken and thickens upon standing). For example, in some embodiments, the synergistic matrix complex includes glyceryl distearate in an amount of from about 2 wt. % to about 5 wt. % and the stearoyl polyoxyl-32 glycerides in an amount of from about 2 wt. % to about 5 wt. %. As another example, in some embodiments, the anhydrous suspension additionally includes Monk fruit sweetener from about 0.1 wt. % to 0.5 wt. %. In yet another example, the anhydrous suspension comprises stearoyl polyoxyl-32 glycerides from about 0.5 wt. % to about 5 wt. %, glyceryl distearate from about 0.5 wt. % to about 5 wt. %, and glycerin from about 5 wt. % to about 10 wt. %. In yet another example, the anhydrous suspension comprises stearoyl polyoxyl-32 glycerides at about 3 wt. %, glyceryl distearate at about 3 wt. %, and glycerin at about 5 wt. %.

These particular example formulations promote a suitable thixotropic effect for maintaining the suspension of APIs. For example, these particular example formulations exhibit particular and unique anti-flocculation, self-emulsification characteristics (e.g., corresponding to the enhanced absorption of APIs), and thixotropic properties, thereby leading to improved homogeneity of the oral pharmaceutical anhydrous suspensions (e.g., that are compatible with nasogastric tube administration). In embodiments, the particular synergistic matrix complex comprising the stearoyl polyoxyl-32 glycerides and glyceryl distearate may also include oils, such as MCT oil or another vegetable oil (e.g., from about 40 wt. % to about 99.5 wt. %). In embodiments, this synergistic matrix complex may also include flavoring agents, such as, for example, Monk fruit sweetener (e.g., or another flavoring agent having an organoleptic property, from about 0.1 wt. % to 0.5 wt. %), or another suitable component.

In these aspects, the stearoyl polyoxyl-32 glycerides and glyceryl distearate suspension enables a rapid dispersion of APIs (e.g., with agitation), and also minimizes sedimentation in the anhydrous suspension. The intermolecular matrix complex of the stearoyl polyoxyl-32 glycerides and glyceryl distearate suspension causes a synergistic effect, which involves a thixotropic co-crystallization and arrangement(s) of carbon chains surrounding one or more oil components (e.g., MCT oil). In addition, the intermolecular matrix complex of the stearoyl polyoxyl-32 glycerides and glyceryl distearate suspension enables the formation of a shear thinning (e.g., promoting the formation of a shear-thinning gel that maintains the suspension of one or more APIs within the suspension). Further, this particular suspension has improved anti-flocculation properties that assists in maintaining the homogeneity of the oral anhydrous pharmaceutical suspension. Further, this particular suspension provides a better texture, based at least in part on the intermolecular matrix complex of the stearoyl polyoxyl-32 glycerides and glyceryl distearate, as well as a better mouth feel.

As another example, the anhydrous pharmaceutical suspension including the stearoyl polyoxyl-32 glycerides and glyceryl distearate may additionally include MCT oil or another oil, preservatives, flavoring agents, a natural oil, or another component, to enhance organoleptically pleasing features of the oral anhydrous pharmaceutical suspension. For example, in some embodiments, the anhydrous pharmaceutical suspension (e.g., including the MCT oil and flavoring agent) may form a thermo-reversible gel that exhibits shear thinning to maintain suspension of the one or more APIs within the anhydrous suspension. For example, a thermos-reversible gel may be a gel that can be melted and reformed in response to a particular temperature. In some aspects, a thermo-reversible gel network can be cross-linked by physical interactions through helix formation (e.g., in gelatin and carrageenan, crystallization in polyethylene, complex formation between polymers such as polyvinyl alcohol and borate). Further, the stearoyl polyoxyl-32 glycerides and glyceryl distearate suspension can also be employed to improve chemical, physical, and microbiologic stability without affecting the therapeutically effective amount of a specific API.

To further illustrate, this anhydrous suspension (e.g., including the stearoyl polyoxyl-32 glycerides from about 0.1 wt. % to about 10 wt. %, glyceryl distearate from about 0.1 wt. % to about 10 wt. %, and one or more of medium chain triglycerides or another oil in an amount of from about 40 wt. % to about 99.5 wt. %, flavoring agent in an amount of from about 0.1 wt. % to about 0.5 wt. %, one or more APIs, and glycerin from about 5 wt. % to about 10 wt. %) enables one or more APIs to remain suspended (e.g., with agitation by stirring or shaking). For example, this synergistic blend decreases the sedimentation velocity of the dispersed APIs by maintaining the viscosity of the suspension at a constant level (based on at least the stearoyl polyoxyl-32 glycerides and glyceryl distearate blend), which minimizes or sufficiently delays the formation of precipitates during administration. The decrease of the sedimentation velocity is caused by the viscosity remaining below a viscosity threshold due to this particular anhydrous suspension (e.g., the intermolecular matrix complex of the stearoyl polyoxyl-32 glycerides and glyceryl distearate suspension).

As such, this synergistic blend results in homogeneity of the APIs in the whole suspension during the period of circulation. Thus, when this anhydrous suspension is used for therapeutic administration to a patient, the viscosity of the synergistic blend allows for an intake of a particular pre-determined amount of API without reducing therapeutic effect.

In some embodiments, each of the stearoyl polyoxyl-32 glycerides and glyceryl distearate may be added to formulate the synergistic blend in amount effective to form a composition having particular thixotropic properties. For example, thixoropy is a time-dependent shear thinning property. As such, certain composition (e.g., gels, fluids) that are thick or viscous under static conditions will flow (e.g., become thinner, less viscous) over time when caused to move, such as being shaken, agitated, shear-stressed, or otherwise stressed. They then take a fixed time to return to a more viscous state. Accordingly, using the particular components for the synergistic blend described herein, each component may be added in a particular amount (e.g., glyceryl distearate in an amount of from about 0.1 wt. % to about 10 wt. % and stearoyl polyoxyl-32 glycerides in an amount of from about 0.1 wt. % to about 10 wt. %) so that the composition exhibits particular thixotropic properties.

As discussed above, in embodiments, the oral anhydrous pharmaceutical suspension disclosed herein may include various components, including, but not limited to, glyceryl distearate NF (e.g., Precirol® ATO 5), stearoyl polyoxyl-32 glycerides NF (e.g., Gelucire® 50/13), medium chain triglycerides NF, vitamin E acetate (DL) USP liquid (1 IU/mg), PUREFRUIT™ select, ascorbyl palmitate NF, glycerin USP (natural), or other components.

In example embodiments, the oral anhydrous pharmaceutical suspension may include glyceryl distearate NF in amount of about 3 wt. %, or from about 0.1 wt. % to about 5 wt. %, or from about 0.5 wt. % to about 4.5 wt. %, or from about 1 wt. % to about 4 wt. %, or from about 1.5 wt. % to about 3.5 wt. %, or from about 1.5 wt. % to about 4 wt. %, or from about 2 wt. % to 4 wt. %, or from about 2.5 wt. % to about 3.5 wt. %, or from about 2.5 wt. % to about 3 wt. %, or from about 2.8 wt. % to about 3.3 wt. %, or from about 2.9 wt. % to about 3.2 wt. %.

In example embodiments, the oral anhydrous pharmaceutical suspension may include stearoyl polyoxyl-32 glycerides NF in an amount of about 3 wt. %, or from about 0.1 wt. % to about 5 wt. %, or from about 0.5 wt. % to about 4.5 wt. %, or from about 1 wt. % to about 4 wt. %, or from about 1.5 wt. % to about 3.5 wt. %, or from about 1.5 wt. % to about 4 wt. %, or from about 2 wt. % to 4 wt. %, or from about 2.5 wt. % to about 3.5 wt. %, or from about 2.5 wt. % to about 3 wt. %, or from about 2.8 wt. % to about 3.3 wt. %, or from about 2.9 wt. % to about 3.2 wt. %.

In example embodiments, the oral anhydrous pharmaceutical suspension may include glyceryl distearate NF in amount from about 0.1 wt. % to about 5 wt. %, stearoyl polyoxyl-32 glycerides NF in an amount from about 0.1 wt. % to about 5 wt. %, and glycerin from about 5 wt. % to about 10 wt. %.

In example embodiments, the oral anhydrous pharmaceutical suspension may include medium chain triglycerides NF (or another oil) in an amount of about 73.5 wt. %, or from about 60 wt. % to about 85 wt. %, or from about 65 wt. % to about 80 wt. %, or from about 68 wt. % to about 75 wt. %, or from about 70 wt. % to about 75 wt. %, or from about 71 wt. % to about 74 wt. %, or from about 71 wt. % to about 75 wt. %, or from about 72 wt. % to about 74 wt. %, from about 73 wt. % to about 74 wt. %, or from about 73 wt. % to about 75 wt. %.

In example embodiments, the oral anhydrous pharmaceutical suspension may include vitamin E (e.g., vitamin E acetate (DL) USP liquid) in amount of about 0.1 wt. %, or from about 0.05 wt. % to about 0.2 wt. %, or from about 0.05 wt. % to about 0.15 wt. %, or from about 0.05 wt. % to about 0.1 wt. %, or from about 0.1 wt. % to about 0.15 wt. %, or from about 0.08 wt. % to about 0.13 wt. %, or from about 0.09 wt. % to about 0.12 wt. %.

In example embodiments, the oral anhydrous pharmaceutical suspension may include a flavoring agent (e.g., PUREFRUIT™ select) in amount of about 0.3 wt. %, or from about 0.1 wt. % to about 0.5 wt. %, or from about 0.2 wt. % to about 0.4 wt. %, or from about 0.15 wt. % to about 0.45 wt. %, or from about 0.25 wt. % to about 0.35 wt. %.

In example embodiments, the oral anhydrous pharmaceutical suspension may include ascorbyl palmitate NF in amount of about 0.1 wt. %, or from about 0.05 wt. % to about 0.2 wt. %, or from about 0.05 wt. % to about 0.15 wt. %, or from about 0.05 wt. % to about 0.1 wt. %, or from about 0.1 wt. % to about 0.15 wt. %, or from about 0.08 wt. % to about 0.13 wt. %, or from about 0.09 wt. % to about 0.12 wt. %.

In example embodiments, the oral anhydrous pharmaceutical suspension may include glycerin USP in an amount of about 20 wt. %, or from about 15 wt. % to about 25 wt. %, or from about 17 wt. % to about 23 wt. %, or from about 18 wt. % to about 22 wt. %, or from about 18 wt. % to about 20 wt. %, or from about 20 wt. % to about 22 wt. %, or from about 19 wt. % to about 21 wt. %, or from about 19.5 wt. % to about 20.5 wt. %.

In example embodiments, the oral anhydrous pharmaceutical suspension can include one or more active pharmaceutical ingredients (APIs). As an example, an API can include one or more of ibuprofen, acetaminophen, chloroquine phosphate, doxycycline hyclate, enrofloxacin, metronidazole, nifedipine, phenoxybenzaine HCL, tretinoin, another type of API, or one or more combinations thereof. As another example, an API that can be taken orally may be added to the oral anhydrous pharmaceutical suspension.

In example embodiments, the oral anhydrous pharmaceutical suspension can include one or more APIs in an amount of from about 0.1 wt. % to about 10 wt. %, or from about 0.1 wt. % to about 8 wt. %, or from about 0.1 wt. % to about 5 wt. %, or from about 0.1 wt. % to about 2 wt. %, or from about 0.1 wt. % to about 1 wt. %.

The embodiments disclosed herein, including the oral anhydrous pharmaceutical suspension and methods corresponding to the oral anhydrous pharmaceutical suspension, may be further understood by reference to the following non-limiting examples.

### EXAMPLES

Example 1 illustrated below is one example embodiment of an oral pharmaceutical anhydrous suspension that exhibits particular thixotropic properties. The table below includes components and an amount (wt. %) of each component.

TABLE 1

| Component | wt. % |
|---|---|
| GLYCERYL DISTEARATE NF (PRECIROL ATO 5) | 3.00% |
| STEAROYL POLYOXYL-32 GLYCERIDES NF | 3.00% |
| (GELUCIRE 50/13) | |
| MEDIUM CHAIN TRIGLYCERIDES NF | 73.50% |
| VITAMIN E ACETATE (DL) USP LIQUID (1 IU/MG) | 0.10% |
| PUREFRUIT ™ SELECT | 0.30% |
| ASCORBYL PALMITATE NF | 0.10% |
| GLYCERIN USP (NATURAL) | 20.00% |
| Totals | 100.00% |

Example 2 includes a synergistic blend of stearoyl polyoxyl-32 glycerides (e.g., in an amount of about 3 wt. %, or from about 0.1 wt. % to about 5 wt. %, or from about 0.5 wt. % to about 4.5 wt. %, or from about 1 wt. % to about 4 wt. %, or from about 1.5 wt. % to about 3.5 wt. %, or from about 1.5 wt. % to about 4 wt. %, or from about 2 wt. % to 4 wt. %, or from about 2.5 wt. % to about 3.5 wt. %, or from about 2.5 wt. % to about 3 wt. %, or from about 2.8 wt. % to about 3.3 wt. %, or from about 2.9 wt. % to about 3.2 wt. %) and glyceryl distearate (e.g., in an amount of about 3 wt. %, or from about 0.1 wt. % to about 5 wt. %, or from about 0.5 wt. % to about 4.5 wt. %, or from about 1 wt. % to about 4 wt. %, or from about 1.5 wt. % to about 3.5 wt. %, or from about 1.5 wt. % to about 4 wt. %, or from about 2 wt. % to 4 wt. %, or from about 2.5 wt. % to about 3.5 wt. %, or from about 2.5 wt. % to about 3 wt. %, or from about 2.8 wt. % to about 3.3 wt. %, or from about 2.9 wt. % to about 3.2 wt. %) for an oral pharmaceutical suspension also including: from about 40 wt. % to 99.5 wt. % of MCT oil (or another oil) and about 0.1 wt. % to 0.5 wt. % of Monk fruit sweetener (or another flavoring agent).

Example 3 includes a synergistic blend of stearoyl polyoxyl-32 glycerides in an amount of about 3 wt. % and glyceryl distearate in an amount of about 3 wt. % for an oral pharmaceutical suspension also including: about 5 wt. % of glycerin, from about 40 wt. % to 99.5 wt. % of MCT oil (or another oil), and about 0.1 wt. % to 0.5 wt. % of Monk fruit sweetener (or another flavoring agent).

Example 4 includes a synergistic blend of stearoyl polyoxyl-32 glycerides in an amount from about 0.1 wt. % to about 5 wt. % and glyceryl distearate in an amount from about 0.1 wt. % to about 5 wt. % for an oral pharmaceutical suspension also including: one or more APIs is present in an amount of from about 0.1 wt. % to about 10 wt. %, from about 40 wt. % to 99.5 wt. % of MCT oil (or another oil), and flavoring agent in an amount of from about 0.1 wt. % to about 0.4 wt. %.

Example 5 includes a synergistic blend of stearoyl polyoxyl-32 glycerides in an amount from about 1.5 wt. % to about 3.5 wt. % and glyceryl distearate in an amount from about 1.5 wt. % to about 3.5 wt. % for an oral pharmaceutical suspension also including: one or more APIs is present in an amount of from about 0.1 wt. % to about 10 wt. %, from about 40 wt. % to 99.5 wt. % of MCT oil (or another oil), vitamin E acetate in an amount of from about 0.05 wt. % to about 0.5 wt. %, and flavoring agent in an amount of from about 0.1 wt. % to about 0.4 wt. %.

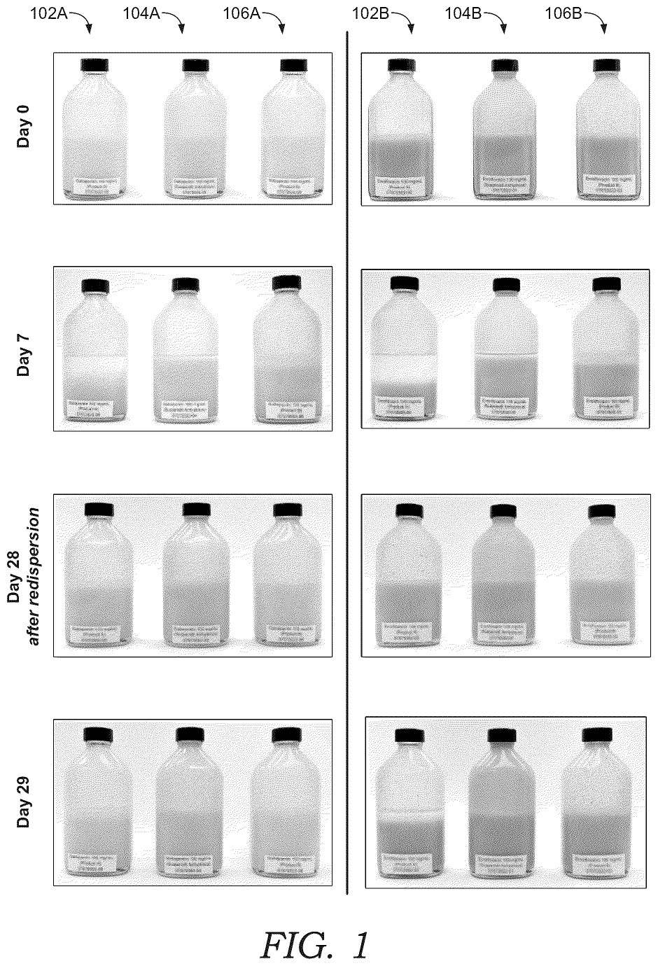

Turning to FIG. 1, the figure depicts results of sedimentation testing to the synergistic blend of stearoyl polyoxyl-32 glycerides and glyceryl distearate, which illustrate the unique physical characteristics of this anhydrous suspension, such as is its capability to improve viscosity, which can also be reduced at a particular length of time via shaking. For example, the improved viscosity illustrated by this discussion allows for rapid redispersion of APIs with mere agitation, thereby minimizing sedimentation (sedimentation including suspended particles being separate from a liquid, e.g., due to gravity over a period of time, and settled on the bottom of a container holding the oral pharmaceutical anhydrous suspension).

For the sedimentation testing illustrated in FIG. 1, a gabapentin 100 mg/mL anhydrous suspension (having a banana cream, yellow color) was prepared using the synergistic blend including stearoyl polyoxyl-32 glycerides and glyceryl distearate, a first prior anhydrous suspension, and a second prior anhydrous suspension. Column 102A depicts the gabapentin 100 mg/mL anhydrous suspension including the first prior anhydrous suspension, column 104A depicts the gabapentin 100 mg/mL anhydrous suspension including the synergistic blend including stearoyl polyoxyl-32 glycerides and glyceryl distearate, and column 106A depicts the gabapentin 100 mg/mL anhydrous suspension including the second prior anhydrous suspension.

Specifically, for the synergistic blend including stearoyl polyoxyl-32 glycerides and glyceryl distearate, each of the components of Table 1, except glycerin, were combined in gabapentin 100 mg/mL anhydrous suspension and heated to 72 degrees Celsius while mixing. Then, this mixture was homogenized and cooled to 38 degrees Celsius on an ice bath while mixing. Glycerin was then added to the mixture. Thereafter, the final mixture continued cooling on an ice bath during mixing until it reached 26.6 degrees Celsius.

Additionally, an enrofloxacin 100 mg/mL anhydrous suspension (having raspberry flavor, pink color) was also prepared using each of the synergistic blend including stearoyl polyoxyl-32 glycerides and glyceryl distearate, the first prior anhydrous suspension, and the second prior anhydrous suspension. Column 102B depicts the enrofloxacin 100 mg/mL anhydrous suspension including the first prior anhydrous suspension, column 104B depicts the enrofloxacin 100 mg/mL anhydrous suspension including the synergistic blend, and column 106B depicts the enrofloxacin 100 mg/mL anhydrous suspension including the second prior anhydrous suspension.

Specifically, for the synergistic blend including stearoyl polyoxyl-32 glycerides and glyceryl distearate, each of the components of Table 1, except glycerin, were combined in enrofloxacin 100 mg/mL anhydrous suspension and heated to 72 degrees Celsius while mixing. Then, this mixture was homogenized and cooled to 38 degrees Celsius on an ice bath while mixing. Glycerin was then added to the mixture. Thereafter, the final mixture continued cooling on an ice bath during mixing until it reached 26.6 degrees Celsius.

With respect to the first prior anhydrous suspension, the first prior anhydrous suspension includes includes glyceryl distearate, polyglyceryl-3 oleate, and medium chain triglycerides. With respect to the second prior anhydrous suspension, the second prior anhydrous suspension includes medium chain triglycerides, lipids, glyceryls, bitter-Bloc™ (a natural flavoring), and Stevia (specifically, TruClear Stevia Plus™).

Further, in a glass container, 10 mL of each suspension was mixed with an equal volume of water by shaking vigorously. Each of the suspensions were placed in a standing position at room temperature for 30 days. On the day 28, each of the glass containers were shaken vigorously with same amount of force and duration, and allowed to settle again. Photographs were taken to the three suspensions throughout the period of the study.

On day 0, the three suspensions (the first of the first prior anhydrous suspension in columns 102A/102B, the second of the synergistic blend in columns 104A/104B, and the third of the second prior anhydrous suspension in columns 106A/106B) exhibited similar sedimentation properties. Notably, a little separation was evident at the top of the suspensions in each of columns 102A/102B and 106A/106B at day 0, as opposed to the synergistic suspension in columns 104A/104B. Further, on day 7, the settling of suspended particles was remarkable for the suspensions in each of columns 102A/102B and 106A/106B. By contrast, the synergistic suspension in columns 104A/104B exhibited only a little separation. By day 29, one day after each of the glass containers were shaken vigorously with same amount of force and duration, the synergistic suspension in columns 104A/104B retained a homogenous suspension with no signs of separation, as opposed to the first prior anhydrous suspension in columns 102A/102B and the second prior anhydrous suspension in columns 106A/106B.

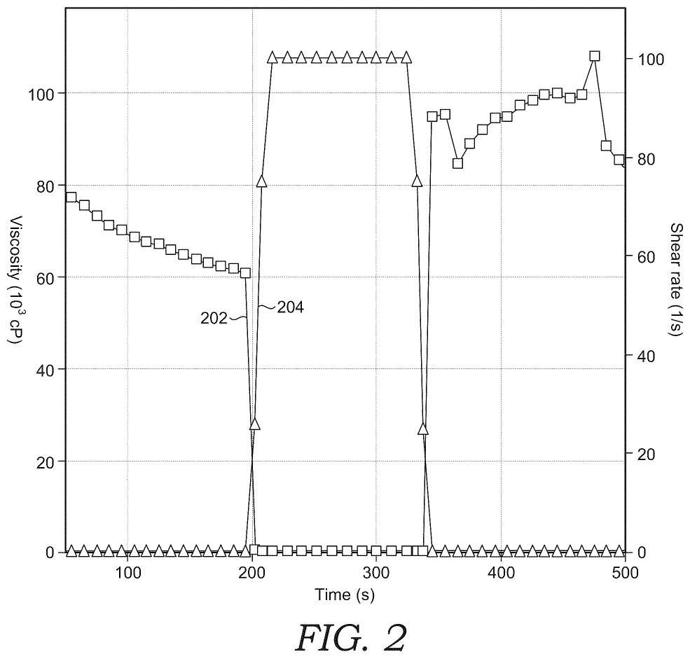

Turning to FIG. 2, graph 200 further illustrates the enhanced thixotropic properties of the synergistic suspension (e.g., the thixotropic properties of the stearoyl polyoxyl-32 glycerides and glyceryl distearate synergistic matrix complex). A thixotropic property refers to the time-dependent, reversible changes in viscosity (resistance to flow). For example, a thixotropic suspension can exhibit stable viscous properties at rest and become fluid when agitated.

To generate the results illustrated by the graph 200, the thixotropic properties of the synergistic suspension (which includes each of the components of Table 1 above) were evaluated. The viscosity instrument used was the Brookfield viscometer Dy-II+Pro. The synergistic suspension was added to a 600 mL beaker and mixed for 5 minutes at 700 rpm. The synergistic suspension was then allowed to rest for 10 minutes. The shear rate applied was 0.1-100 1/s.

The line 202 illustrates the change of viscosity of the synergistic suspension and line 204 illustrates the change of shear rate of the synergistic suspension.

When low shear (0.1/s) was applied for the first 200 seconds, the suspension maintained its viscosity at rest (depicted by line 204). Viscosity dropped to around 0 cp (depicted by line 202) immediately after shear rate was increased (100/s). At a later time, when shear was back to low rate (0.1/s), the suspension regained its viscosity at rest within seconds. In this way, the thixotropic properties of the synergistic suspension can be described as: thinning when shaken and thickening upon standing. For example, thinning when shaken is a particular property that allows for a rapid redispersion of an API, thereby ensuring dose uniformity for suitable administration. Further, the thinning when shaken also facilitates a more precise and accurate measurement for administration of each dose. Additionally, the thickening upon standing property eliminates or minimizes sedimentation of an API once the suspension settles, thereby ensuring physical stability of the suspension.

Additionally, physical properties of a suspension including the stearoyl polyoxyl-32 glycerides and glyceryl distearate blend can affect the one or more particular particle sizes of the one or more APIs when mixed with the blend. For example, the particle size of the APIs in the suspensions can affect uniformity, stability, and bioavailability of the drugs within the suspensions when administered to patients. To illustrate, smaller and uniform particle sizes of APIs in suspensions are less likely to agglomerate and separate, thereby contributing to the physical stability of a suspension. In addition, smaller particle sizes are more easily dispersed, which results in improved uniformity of the suspensions.

To illustrate, in another example experiment, compounded anhydrous oral suspensions were prepared for ibuprofen 40 mg/mL (one example of an API) in the stearoyl polyoxyl-32 glycerides and glyceryl distearate blend (which includes each of the components of Table 1 above). Two other blends using an industry standard aqueous suspension and the second prior anhydrous suspension (the second prior anhydrous suspension corresponding to columns 106A/106B of FIG. 1) were also prepared with ibuprofen 40 mg/mL for comparison of uniformity, stability, and bioavailability with the stearoyl polyoxyl-32 glycerides and glyceryl distearate blend. In this way, differences in the physical properties of the stearoyl polyoxyl-32 glycerides and glyceryl distearate blend corresponding to ibuprofen particle size could be identified.

The test samples for the ibuprofen 40 mg/mL with each of the stearoyl polyoxyl-32 glycerides and glyceryl distearate blend, the second prior anhydrous suspension, and the industry standard aqueous suspension (Motrin Infant Ibuprofen 40 mg/mL) were prepared by shaking each of the containers vigorously for about 30 seconds. The analysis consisted in dispersing 10 g of each suspension in 15 mL of simulated gastric fluid, followed by 1 hour of stirring. The particle size detector used was the Saturn DigiSizer II 5205 V1.04. Sampling was performed to conform to PTA SOP 001/44053/00, and the tests were run in accordance with ISO 13320. Table 2 below includes particle size information with respect to the industry standard aqueous suspension.

TABLE 2

| | Particle Size Diameter (μm) | | | |
|---|---|---|---|---|
| Anhydrous Suspensions | 90% | 50% | 10% | Mean ± Std Dev |
| Ibuprofen (Reference | 152.004 | 76.430 | 28.231 | 83.402 ± 0.492 |
| industry standard | | | | |
| suspension) | | | | |

In this example experiment, most of the ibuprofen particles in the stearoyl polyoxyl-32 glycerides and glyceryl distearate blend presented a diameter of about 57.1 μm-75.6 μm. These particle sizes were more than two times smaller than the particle sizes for the second prior anhydrous suspension.

Specifically, most of the ibuprofen particles in the second prior anhydrous suspension presented a much larger diameter of about 192.7 μm-255.3 μm.

Based on this example experiment, the stearoyl polyoxyl-32 glycerides and glyceryl distearate blend contributed to a small particle size diameter for ibuprofen, thereby illustrating/indicating the enhanced uniformity, stability, and bioavailability attributed by the stearoyl polyoxyl-32 glycerides and glyceryl distearate blend. The table below summaries the observed particle sizes for this example experiment.

TABLE 3

| | Particle Size Diameter (μm) | | | |
|---|---|---|---|---|
| Anhydrous Suspensions | 90% | 50% | 10% | Mean ± Std Dev |
| Ibuprofen 40 mg/mL | 75.607 | 57.148 | 5.282 | 49.876 ± 0.513 |
| (stearoyl polyoxyl- | | | | |
| 32 glycerides and | | | | |
| glyceryl distearate | | | | |
| blend) | | | | |
| Ibuprofen 40 mg/mL | 73.228 | 36.356 | 9.891 | 38.652 ± 0.142 |
| (Reference industry | | | | |
| standard aqueous | | | | |
| suspension) | | | | |
| Ibuprofen 40 mg/mL | 255.273 | 192.657 | 96.671 | 1830.034 |
| (second prior | | | | |
| anhydrous suspension) | | | | |

In yet another example experiment, the content uniformity of the stearoyl polyoxyl-32 glycerides and glyceryl distearate blend including an API dispersed in the suspension was analyzed. Stated differently, the suspension tested included an insoluble API dispersed in a liquid medium (suspending vehicle).

The content uniformity can be defined as the consistency in the amount of the API among dosage units. This experiment, described herein further, illustrates that the stearoyl polyoxyl-32 glycerides and glyceryl distearate was uniform in content, such that each dose was equivalent in the amount of APIs. Content uniformity within a suspension can dependent on the characteristics of the suspending vehicle (i.e. the content uniformity depends upon the characteristics of the stearoyl polyoxyl-32 glycerides and glyceryl distearate blend). In different suspending vehicles that are too viscous, the APIs do not easily disperse. In different suspending vehicles that are too thin, the APIs settle at the bottom of the container.

The evaluation of the content uniformity for the stearoyl polyoxyl-32 glycerides and glyceryl distearate blend was divided in two stages: 1. Elaboration of the stearoyl polyoxyl-32 glycerides and glyceryl distearate suspensions according to the corresponding PCCA Formulas (see Table 4 below). (For example, some formulas were prepared in the PCCA laboratory using the Flacktek Speedmixer® (indicated in Table 4 below).) 2. Potency testing by Ultra-Performance Liquid Chromatography (UPLC) assay.

TABLE 4

| stearoyl polyoxyl-32 glycerides and | PCCA | Mean % Recovery |
|---|---|---|
| glyceryl distearate suspension | Formula | Room Temperature |
| Chloroquine Phosphate 100 mg/5 mL | 14216 | 103.00 |
| Doxycycline Hyclate 50 mg/mL | 14222 | 100.97 |
| Doxycycline Hyclate 100 mg/mL | 14223 | 90.51 |
| Doxycycline Hyclate 100 mg/mL | | 103.00 |
| (Flacktek) | | |
| Enrofloxacin 10 mg/mL (Vet) | 14224 | 108.00 |
| Enrofloxacin 100 mg/mL (Vet) | 14225 | 106.00 |
| Metronidazole 50 mg/mL | 14243 | 108.71 |
| Metronidazole 50 mg/mL (Alternate) | 14244 | 101.24 |
| Nifedipine 4 mg/mL | 14247 | 102.00 |
| Phenoxybenzamine HCl 2 mg/mL | 14252 | 98.00 |
| Tretinoin 10 mg/mL | 14260 | 95.00 |
| Tretinoin 10 mg/mL (Flacktek) | | 93.80 |

The test samples, corresponding to Table 4 above, were stored at room temperature and were analysed by the analytical laboratory in the PCCA Research & Development department or by the Eagle Analytical Services Inc. For each sample, ten sampling points were taken for analysis and the value reported is the average of all sampling points.

The potency testing of all three aliquots per sample, corresponding to Table 4 above, showed that none of the anhydrous suspensions including the stearoyl polyoxyl-32 glycerides and glyceryl distearate blend were outside of the 90.0%-110.0% potency specification (USP <621>chapter: Chromatography). As such, the disclosed vehicle of the stearoyl polyoxyl-32 glycerides and glyceryl distearate blend enhances the content uniformity of an API (e.g., of an individual API in variable strengths). This also illustrates that the stearoyl polyoxyl-32 glycerides and glyceryl distearate formulations (e.g., glyceryl distearate in an amount of from about 0.1 wt. % to about 10 wt. % and stearoyl polyoxyl-32 glycerides in an amount of from about 0.1 wt. % to about 10 wt. %; glyceryl distearate in an amount of from about 2.5 wt. % to about 3.5 wt. % and stearoyl polyoxyl-32 glycerides in an amount of from about 2.5 wt. % to about 3.5 wt. %) had content uniformity.

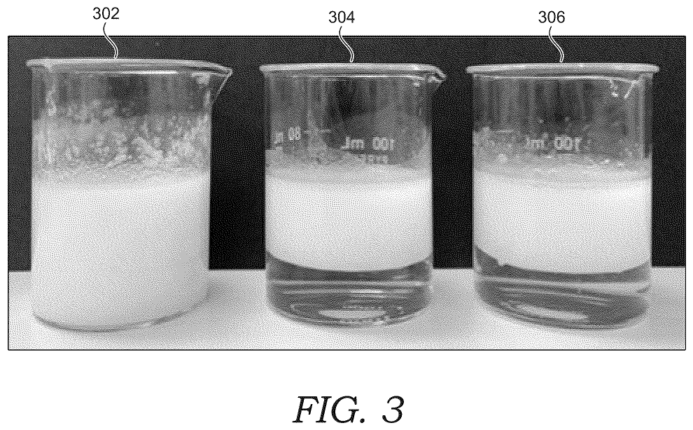

Turning to FIGS. 3-5, the figures depict another test that helps illustrate other particular physical characteristics of the disclosed anhydrous suspension related to self-emulsifying properties of the suspension.

The anhydrous suspension including the stearoyl polyoxyl-32 glycerides and glyceryl distearate blend is a Self-Emulsifying Drug Delivery Systems (SEDDS) capable of creating a spontaneous emulsion when in contact with water. This spontaneous emulsion can release an API from the oral base, thereby increasing drug solubility, dispersibility, absorption and bioavailability. The anhydrous suspension discussed herein can include particular surfactants within an anhydrous oral vehicle, such that it easily mixes with water, juice, or another aqueous solution, without further additional surfactants. The self-emulsifying properties of the oral pharmaceutical anhydrous suspension can improve or enhance the dispersion of water-soluble APIs and drug release of lipophilic drugs, for instance.

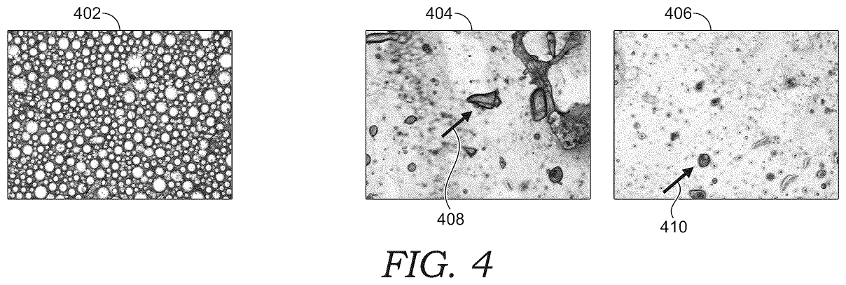

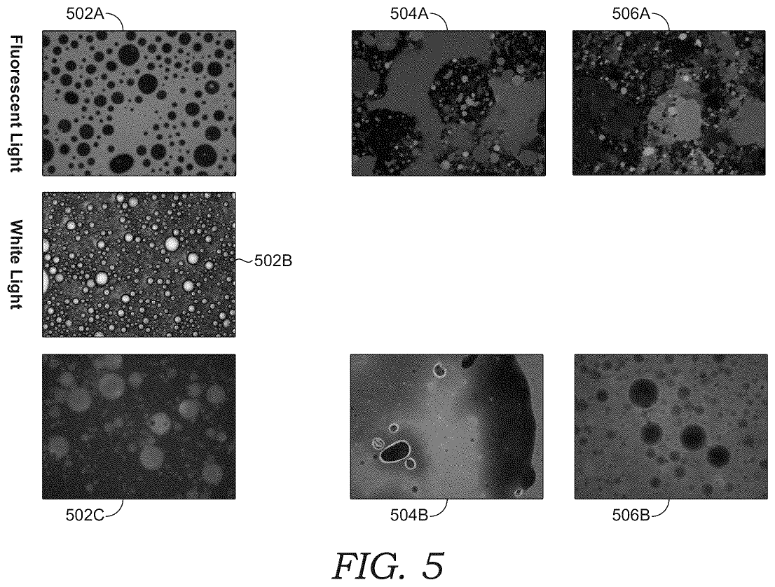

In FIGS. 3-5, the disclosed anhydrous suspension was compared to prior anhydrous suspension products. Testing of the self-emulsifying properties of the stearoyl polyoxyl-32 glycerides and glyceryl distearate blend and other prior anhydrous suspension products was conducted, and evaluation of the self-emulsifying properties was performed using both microscopic evaluation, corresponding to FIG. 4, and fluorescence microscopy, corresponding to FIG. 5.

Specifically, a metronidazole 50 mg/mL anhydrous suspension was prepared using the stearoyl polyoxyl-32 glycerides and glyceryl distearate suspension (the suspension including the components of Table 1) in one beaker and two other prior anhydrous suspension products for the other two beakers. Beaker 302 includes the metronidazole 50 mg/mL anhydrous suspension prepared with the stearoyl polyoxyl-32 glycerides and glyceryl distearate suspension. Additionally, beaker 304 includes the metronidazole 50 mg/mL anhydrous suspension prepared with a first prior anhydrous suspension (the first prior anhydrous suspension corresponding to columns 102A/102B of FIG. 1), and beaker 306 includes the metronidazole 50 mg/mL anhydrous suspension prepared with the second prior anhydrous suspension (the second prior anhydrous suspension corresponding to columns 106A/106B of FIG. 1).

Each of the three suspensions were mixed in water at 1:1 ratio (v/v), and these suspensions within the beakers 302-306 are illustrated in FIG. 3. Furthermore, a sample of each suspension was observed under a light microscope (reproduced in FIG. 4) with a total magnification of 100x for evaluation of the droplet size formation. When the samples were observed under the microscope at 4× magnification, the suspension in the beaker 302 (including the metronidazole 50 mg/mL anhydrous suspension prepared with the stearoyl polyoxyl-32 glycerides and glyceryl distearate suspension) resulted in a homogeneous dispersion, as illustrated by its corresponding microscopic evaluation 402 in FIG. 4. By contrast, the suspension in the beaker 306 (including the metronidazole 50 mg/mL anhydrous suspension prepared with the second prior anhydrous suspension) displayed aggregation of metronidazole, as illustrated by its corresponding microscopic evaluation 404 (indicated via arrow 408). Furthermore, the suspension in beaker 306 (including the metronidazole 50 mg/mL anhydrous suspension prepared with the second prior anhydrous suspension) also displayed a separation of the water and oily phases, as illustrated by its additional corresponding microscopic evaluation 406 (indicated via arrow 410).

When each of the metronidazole 50 mg/mL anhydrous suspensions were mixed with water, the beaker 302 including the metronidazole 50 mg/mL anhydrous suspension prepared with the stearoyl polyoxyl-32 glycerides and glyceryl distearate suspension formed a uniform emulsion, indicating beneficial drug absorption. For the other two beakers 304 and 306, the suspensions using the prior anhydrous suspensions were immiscible with water, and formed two separate phases (indicated via arrows 308A/308B and 310A/310B). As illustrated by FIGS. 3-4, the SEDDS in the stearoyl polyoxyl-32 glycerides and glyceryl distearate suspension allow for the formation of a uniform emulsion, characterized by small and homogeneous droplets illustrated by microscopic evaluation 402. As a result, the self-emulsifying properties of the stearoyl polyoxyl-32 glycerides and glyceryl distearate suspension can promote increased API solubility, dispersibility, absorption, and bioavailability.

To generate the fluorescent images 502A, 504A, and 506A, which were generated to further characterize the self-emulsifying properties of the stearoyl polyoxyl-32 glycerides and glyceryl distearate suspension, fluorescence microscopy at 4×objective lens and fluorescein sodium 1 wt. % was used for each of the stearoyl polyoxyl-32 glycerides and glyceryl distearate suspension, the first prior anhydrous suspension, and the second prior anhydrous suspension. For example, fluorescent light and the 4×objective lens for fluorescein sodium 1 wt. % in the stearoyl polyoxyl-32 glycerides and glyceryl distearate suspension was used to generate the fluorescent image 502A, and white light was used to generate fluorescent image 502B. Additionally, fluorescent light and the 4×objective lens for fluorescein sodium 1 wt. % in the first prior anhydrous suspension and second prior anhydrous suspension was used to generate the fluorescent images 504A (corresponding to the first prior anhydrous suspension) and 506A (corresponding to the second prior anhydrous suspension).

To generate the fluorescent images 502C (corresponding to the stearoyl polyoxyl-32 glycerides and glyceryl distearate suspension), 504B (corresponding to the first prior anhydrous suspension), and 506B (corresponding to the second prior anhydrous suspension), fluorescence microscopy (fluorescent light) at 10×objective lens for curcumin 1 wt. % was used for each of the suspensions.

The fluorescent images 502A, 502B, 502C, 504A, 504B, 506A, and 506B allow for the evaluation of the distribution pattern of the hydrophilic substance fluorescein sodium (high solubility), and also the lipophilic substance curcumin (low solubility and low permeability), by way of comparison of each of the suspensions. Each of the three suspensions were gently mixed with water at 1:1 ratio (v/v). Each of the images were taken using a Nikon Eclipse TS100 inverted phase microscope coupled with the NIS-Elements imaging software.

Fluorescein sodium and curcumin were used to represent the hydrophilic and lipophilic APIs that are commonly used in clinical practice. The results illustrated in FIG. 5 show that, upon mixing the stearoyl polyoxyl-32 glycerides and glyceryl distearate suspension with water at 1:1 ratio (v/v), the stearoyl polyoxyl-32 glycerides and glyceryl distearate suspension exhibited particular self-emulsifying properties by creating an emulsion, i.e. dispersed lipophilic droplets in an aqueous continuous phase.

With respect to the solutions including the fluorescein sodium 1 wt. % and the stearoyl polyoxyl-32 glycerides and glyceryl distearate suspension, the fluorescein sodium was soluble in both the water and the oil droplets, due to the uniform emulsion via the stearoyl polyoxyl-32 glycerides and glyceryl distearate suspension. For example, this uniform emulsion is illustrated in fluorescent images 502A and 502B.

In contrast, with respect to the solutions including the fluorescein sodium 1 wt. % and the other prior anhydrous suspensions, each of the fluorescent images 504A and 506A indicated a lack of uniform emulsion being formed. Further, most of the fluorescein sodium within the first and second prior anhydrous suspensions were soluble in water, and there was separation between the water and oily phases within the first and second prior anhydrous suspensions, as illustrated by fluorescent images 504A and 506A.

In addition, with respect to the solutions including the curcumin and the stearoyl polyoxyl-32 glycerides and glyceryl distearate suspension, the insoluble curcumin was enclosed inside the oil droplets, and a uniform emulsion was formed for the stearoyl polyoxyl-32 glycerides and glyceryl distearate suspension, as illustrated in fluorescent image 502C.

In contrast, with respect to the solutions including the curcumin and the other prior anhydrous suspensions, the curcumin mainly dispersed in the oil phase and did not disperse in the water. Furthermore, there was separation between the oil and water phases, as illustrated in fluorescent images 504B and 506B.

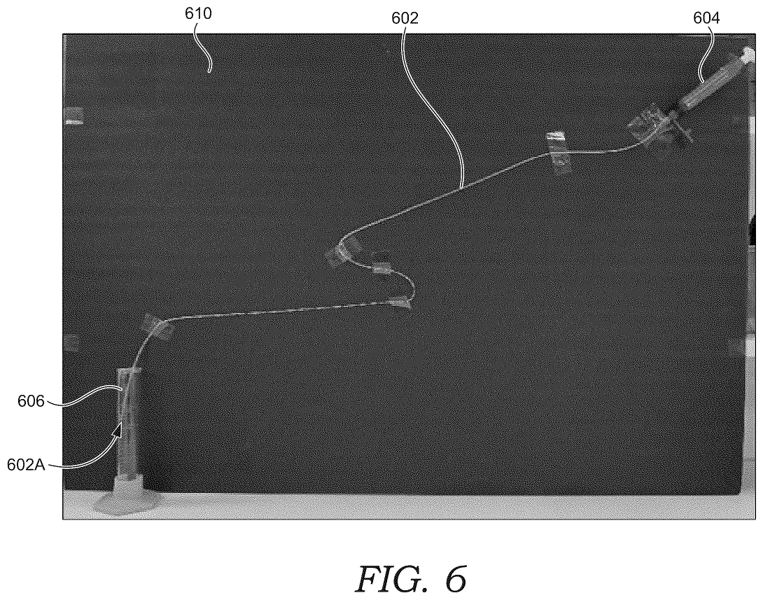

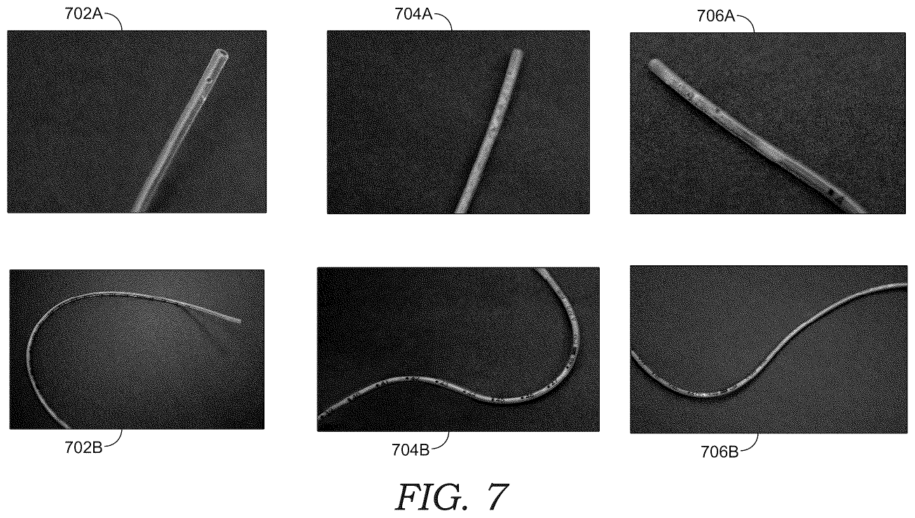

Turning to FIGS. 6-7, example images in these figures depict various nasogastric feeding tubes. Specifically, FIG. 6 illustrates a nasogastric feeding tube setting for generating the images 702A-702B, 704A-704B, and 706A-706B of FIG. 7.

Nasogastric feeding tubes are commonly used in hospitalized patients when enteral nutrition is required. One problem patients and healthcare providers encounter includes tube clogging, which inhibits the delivery of nutritional support. As illustrated by FIGS. 6-7 and further discussed herein, the stearoyl polyoxyl-32 glycerides and glyceryl distearate suspension can reduce the likelihood of compounded metronidazole anhydrous suspensions that can block or leave residues in nasogastric feeding tubes (e.g., for pediatrics).

To generate the images 702A-702B, 704A-704B, and 706A-706B, and with reference to FIG. 6, a pediatrics nasogastric feeding tube 602 was taped to a black surface 610 and placed downwards for gravitational effect. An oral syringe 604 was placed at the entrance of the nasogastric feeding tube 602, and a graduated cylinder 606 was placed at the exit 602A of the nasogastric feeding tube 602 to collect the suspension injected from the syringe 604.

To remove any water residues from the nasogastric feeding tube 602, the nasogastric feeding tube 602 was flushed with 5 mL of deionized water and then 3 mL of air. Metronidazole 50 mg/mL oral suspensions were prepared using the stearoyl polyoxyl-32 glycerides and glyceryl distearate blend (for the subsequent generation of images 702A-702B), the first prior anhydrous suspension (for the subsequent generation of images 704A-704B), and the second prior anhydrous suspension (for the subsequent generation of images 706A-706B).

The potency of the three different oral suspensions (the metronidazole 50 mg/mL oral suspension using the stearoyl polyoxyl-32 glycerides and glyceryl distearate blend, the metronidazole 50 mg/mL oral suspension using the first prior anhydrous suspension, and the metronidazole 50 mg/mL oral suspension using the second prior anhydrous suspension) was tested using High-Performance Liquid Chromatography (HPLC). Specifically, a volume of 5 mL of the metronidazole 50 mg/mL oral suspension in the stearoyl polyoxyl-32 glycerides and glyceryl distearate blend was delivered through the oral syringe 604 and pushed through the nasogastric feeding tube 602 at a rate of 5 mL/25 s. The oral suspension was collected into the graduated cylinder 606 for potency testing by HPLC. The nasogastric feeding tube 602 was then flushed with 10 mL of deionized water, and the test was repeated once again.

Following these two test injections, the nasogastric feeding tube 602 was allowed to dry until the next day. Finally, the tube was checked for any signs of residues or clogging, and photographs were taken to generate images 702A-702B. This procedure was repeated for the metronidazole 50 mg/mL oral suspension using the first prior anhydrous suspension to generate images 704A-704B, and for the metronidazole 50 mg/mL oral suspension using the second prior anhydrous suspension to generate images 706A-706B.

On the day after the recovery test, each of the images 702A-702B, 704A-704B, and 706A-706B were generated. Specifically, the images 702A, 704A, and 706A are close-up images of the exit 602A of the nasogastric feeding tube 602, and the images 702B, 704B, and 706B are extended images of the nasogastric feeding tube 602. With respect to the images 702A-702B, it was observed that there were no significant drug residues throughout the nasogastric tube 602, and no significant drug residues at the the exit 602A of the nasogastric feeding tube 602—for the metronidazole 50 mg/mL oral suspension in the stearoyl polyoxyl-32 glycerides and glyceryl distearate blend. By contrast, there was visible accumulation of drug residues for the the metronidazole 50 mg/mL oral suspensions including the first and second prior anhydrous oral suspensions, which are illustrated in images 704A-704B and 706A and 706B.

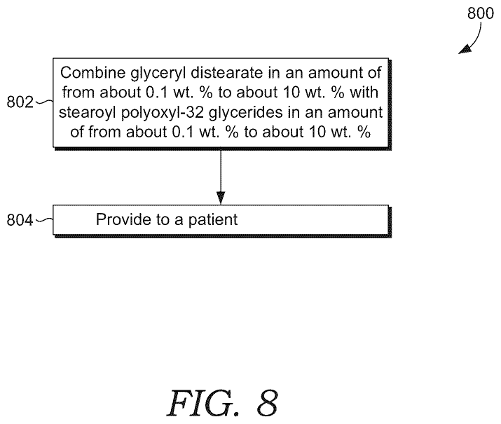

FIG. 8 depicts an example flowchart for method 800 for the manufacturing and usage of the stearoyl polyoxyl-32 glycerides and glyceryl distearate suspension. It should be noted that, although the term “step” can be used herein with respect to FIG. 8 to connote different elements of the method described, this term should not be interpreted as implying any particular order among or between various steps discussed herein with reference to FIG. 8. For example, in other embodiments, the one or more APIs are added at a different step.

At step 802, glyceryl distearate in an amount of from about 0.1 wt. % to about 10 wt. % is combined with stearoyl polyoxyl-32 glycerides in an amount of from about 0.1 wt. % to about 10 wt. %. Additionally, in some embodiments, one or more of the following are combined with the are combined with the stearoyl polyoxyl-32 glycerides and glyceryl distearate blend: an API, medium chain triglycerides in an amount of from about 40 wt. % to about 99.5 wt. %; flavoring agent in an amount of from about 0.1 wt. % to about 0.5 wt. %, from about 5 wt. % to about 25 wt. %, ascorbyl palmitate NF in amount from about 0.05 wt. % to about 0.15 wt. %, vitamin E acetate in amount from about 0.05 wt. % to about 0.2 wt. %, another component, or one or more combinations thereof. At step 804, the stearoyl polyoxyl-32 glycerides and glyceryl distearate suspension (including the one or more components of step 804) is provided to a patient (e.g., for oral usage, via a nasogastric tube, syringe, etc.).

The subject matter of the present technology is described with specificity herein to meet statutory requirements. However, the description itself is not intended to limit the scope of this disclosure. Rather, the inventors have contemplated that the claimed or disclosed subject matter might also be embodied in other ways, to include different steps or combinations of steps similar to the ones described in this document, in conjunction with other present or future technologies.

Some example aspects of the technology that may be practiced from the forgoing disclosure include the following:

Aspect 1: An anhydrous suspension comprising: glyceryl distearate in an amount of from about 0.1 wt. % to about 10 wt. %; stearoyl polyoxyl-32 glycerides in an amount of from about 0.1 wt. % to about 10 wt. %; and one or more active pharmaceutical ingredients (APIs).

Aspect 2: The anhydrous suspension of Aspect 1, wherein the glyceryl distearate is present in an amount of from about 2 wt. % to about 5 wt. %.

Aspect 3: The anhydrous suspension of any of Aspects 1-2, wherein the glyceryl distearate is present in an amount of from about 2.5 wt. % to about 3.5 wt. %.

Aspect 4: The anhydrous suspension of any of Aspects 1-3, wherein the stearoyl polyoxyl-32 glycerides are present in an amount of from about 2 wt. % to about 5 wt. %.

Aspect 5, The anhydrous suspension of any of Aspects 1-4, wherein the stearoyl polyoxyl-32 glycerides are present in an amount of from about 2.5 wt. % to about 3.5 wt. %.

Aspect 6: The anhydrous suspension of any of Aspects 1-5, wherein the one or more APIs is present in an amount of from about 0.1 wt. % to about 10 wt. %.

Aspect 7: The anhydrous suspension of any of Aspects 1-6, wherein the one or more APIs is selected from the group consisting of ibuprofen, acetaminophen, chloroquine phosphate, doxycycline hyclate, enrofloxacin, metronidazole, nifedipine, phenoxybenzaine HCL, or tretinoin.

Aspect 8: The anhydrous suspension of any of Aspects 1-7, further comprising oil in an amount of from about 40 wt. % to about 99.5 wt. %.

Aspect 9: The anhydrous suspension of any of Aspects 1-8, further comprising a flavoring agent in an amount of from about 0.1 wt. % to about 0.5 wt. %.

Aspect 10: The anhydrous suspension of any of Aspects 1-9, wherein the anhydrous suspension exhibits thixotropic properties.

Aspect 11: The anhydrous suspension of any of Aspects 1-10, wherein a viscosity of the anhydrous suspension decreases over time as shear rate is increased.

Aspect 12: The anhydrous suspension of any of Aspects 1-11, wherein increasing the shear rate comprises one or more of agitation, shaking, shear-stress, or other stress.

Aspect 13: The anhydrous suspension of any of Aspects 1-10, wherein the viscosity of the anhydrous suspension increases over time as shear rate is decreased.

Aspect 14: An anhydrous suspension comprising: medium chain triglycerides in an amount of from about 40 wt. % to about 99.5 wt. %; flavoring agent in an amount of from about 0.1 wt. % to about 0.5 wt. %; glyceryl distearate in an amount of from about 0.1 wt. % to about 10 wt. %; stearoyl polyoxyl-32 glycerides in an amount of from about 0.1 wt. % to about 10 wt. %; and one or more active pharmaceutical ingredients (APIs), wherein the anhydrous suspension exhibits a thixotropic flow.

Aspect 15: The anhydrous suspension of Aspect 14, wherein the glyceryl distearate is present in an amount of from about 2 wt. % to about 5 wt. %.

Aspect 16: The anhydrous suspension of any of Aspects 14-15, wherein the glyceryl distearate is present in an amount of from about 2.5 wt. % to about 3.5 wt. %.

Aspect 17: The anhydrous suspension of any of Aspects 14-16, wherein the stearoyl polyoxyl-32 glycerides are present in an amount of from about 2 wt. % to about 5 wt. %.

Aspect 18: The anhydrous suspension of any of Aspects 14-17, wherein the stearoyl polyoxyl-32 glycerides are present in an amount of from about 2.5 wt. % to about 3.5 wt. %.

Aspect 19: The anhydrous suspension of any of Aspects 14-18, wherein the one or more APIs is present in an amount of from about 0.1 wt. % to about 10 wt. %.

Aspect 20: The anhydrous suspension of any of Aspects 14-19, wherein the medium chain triglycerides (MCT) are MCT oil or a vegetable oil.

Aspect 21: The anhydrous suspension of any of Aspects 14-20, wherein the flavoring agent is Monk fruit sweetener.

Aspect 22: The anhydrous suspension of any of Aspects 14-21, wherein the anhydrous suspension forms a thermo-reversible gel and exhibits shear thinning to maintain the one or more APIs suspended within the anhydrous suspension.

Aspect 23: The anhydrous suspension of any of Aspects 14-22, wherein a viscosity of the anhydrous suspension decreases over time as shear rate is increased.

Aspect 24: The anhydrous suspension of any of Aspects 14-23, wherein increasing the shear rate comprises one or more of agitation, shaking, shear-stress, or other stress.

Aspect 25: The anhydrous suspension of any of claims 14-24, wherein the viscosity of the anhydrous suspension increases over time as shear rate is decreased.

Aspect 26: A method for using an anhydrous suspension comprising: combining glyceryl distearate in an amount of from about 0.1 wt. % to about 10 wt. %, stearoyl polyoxyl-32 glycerides in an amount of from about 0.1 wt. % to about 10 wt. %, and one or more active pharmaceutical ingredients (APIs); and providing the anhydrous suspension for oral use by a patient.

Aspect 27: A method of manufacturing an anhydrous suspension comprising: combining glyceryl distearate in an amount of from about 0.1 wt. % to about 10 wt. %, stearoyl polyoxyl-32 glycerides in an amount of from about 0.1 wt. % to about 10 wt. %, and one or more active pharmaceutical ingredients (APIs).

Aspect 28: A method of manufacturing an anhydrous suspension having thixotropic properties, the method comprising: combining glyceryl distearate and stearoyl polyoxyl-32 gycerides in an amount effective for the anhydrous suspension to exhibit the thixotropic properties.

Aspect 29: A composition comprising: glyceryl distearate; and stearoyl polyoxyl-32 glycerides, wherein the glyceryl distearate and the stearoyl polyoxyl-32 glycerides are added in an amount effective for the composition to exhibit thixotropic properties.

What is claimed is:

1. An anhydrous suspension comprising:
glyceryl distearate in an amount of from about 0.1 wt. % to about 10 wt. %;
stearoyl polyoxyl-32 glycerides in an amount of from about 0.1 wt. % to about 10 wt. %; and
one or more active pharmaceutical ingredients (APIs).

2. The anhydrous suspension of claim 1, wherein the glyceryl distearate is present in an amount of from about 2 wt. % to about 5 wt. %.

3. The anhydrous suspension of claim 1, wherein the glyceryl distearate is present in an amount of from about 2.5 wt. % to about 3.5 wt. %.

4. The anhydrous suspension of claim 1, wherein the stearoyl polyoxyl-32 glycerides are present in an amount of from about 2.5 wt. % to about 3.5 wt. %.

5. The anhydrous suspension of claim 1, wherein the one or more APIs is present in an amount of from about 0.1 wt. % to about 10 wt. %.

6. The anhydrous suspension of any of claim 1, wherein the one or more APIs corresponds to one or more of ibuprofen, acetaminophen, chloroquine phosphate, doxycycline hyclate, enrofloxacin, metronidazole, nifedipine, phenoxybenzaine HCL, and tretinoin.

7. The anhydrous suspension of claim 1, further comprising oil in an amount of from about 40 wt. % to about 99.5 wt. %.

8. The anhydrous suspension of claim 1, further comprising a flavoring agent in an amount of from about 0.1 wt. % to about 0.5 wt. %.

9. The anhydrous suspension of claim 1, wherein the anhydrous suspension exhibits thixotropic properties.

10. The anhydrous suspension of claim 1, wherein a viscosity of the anhydrous suspension decreases over time as shear rate is increased.

11. The anhydrous suspension of claim 10, wherein increasing the shear rate comprises one or more of agitation, shaking, and shear-stress.

12. The anhydrous suspension of claim 10, wherein the viscosity of the anhydrous suspension increases over time as shear rate is decreased.

13. An anhydrous suspension comprising:
medium chain triglycerides (MCT) in an amount of from about 40 wt. % to about 99.5 wt. %;
a flavoring agent in an amount of from about 0.1 wt. % to about 0.5 wt. %;
glyceryl distearate in an amount of from about 0.1 wt. % to about 10 wt. %;
stearoyl polyoxyl-32 glycerides in an amount of from about 0.1 wt. % to about 10 wt. %; and
one or more active pharmaceutical ingredients (APIs), wherein the anhydrous suspension exhibits a thixotropic flow.

14. The anhydrous suspension of claim 13, wherein the stearoyl polyoxyl-32 glycerides are present in an amount of from about 2 wt. % to about 5 wt. %.

15. The anhydrous suspension of claim 13, wherein the stearoyl polyoxyl-32 glycerides are present in an amount of from about 2.5 wt. % to about 3.5 wt. %.

16. The anhydrous suspension of claim 13, wherein the one or more APIs is present in an amount of from about 0.1 wt. % to about 5 wt. %.

17. The anhydrous suspension of claim 13, wherein the medium chain triglycerides include one or more of an MCT oil or a vegetable oil.

18. The anhydrous suspension of claim 13, wherein the flavoring agent is Monk fruit sweetener.

19. The anhydrous suspension of claim 13, wherein the anhydrous suspension forms a thermo-reversible gel and exhibits shear thinning to maintain the one or more APIs suspended within the anhydrous suspension.

20. A method for using an anhydrous suspension comprising:
combining glyceryl distearate in an amount of from about 0.1 wt. % to about 10 wt. %, stearoyl polyoxyl-32 glycerides in an amount of from about 0.1 wt. % to about 10 wt. %, and one or more active pharmaceutical ingredients (APIs); and
providing the anhydrous suspension for oral use by a patient.
